(19) United States
(12) Patent Application Publication
(10) Pub. No.: US 2020/0306408 A1
(43) Pub. Date: Oct. 1, 2020

(54) CYCLODEXTRIN CONTAINING FRESHENING COMPOSITION

(71) Applicant: The Procter & Gamble Company, Cincinnati, OH (US)

(72) Inventors: Michael David O'Young MUI, Cincinnati, OH (US); Christine Marie READNOUR-GOUGE, Ft. Mitchell, KY (US); Melissa Jane WENE, Lebanon, OH (US); Gregory Scot MIRACLE, Liberty Township, OH (US)

(21) Appl. No.: 16/367,419

(22) Filed: Mar. 28, 2019

(51) Int. Cl. A61L 9/14; A61L 2/22

(52) U.S. Cl. CPC A61L 9/145; A61L 2/22

(57) ABSTRACT

A freshening composition and a consumer product that includes the freshening composition are provided. The freshening composition includes cyclodextrin; a silicone anti-foam agent, and a liquid carrier. The freshening composition includes a weight ratio of silicone anti-foam agent to cyclodextrin of less than 0.00375:1. A consumer product may include the freshening composition disposed in a spray dispenser.

### FIELD

The present invention relates to freshening compositions for the air and surfaces, and more particularly, relates to freshening compositions comprising cyclodextrin and a low-level of a silicone anti-foam agent.

### BACKGROUND

Freshening compositions comprising perfumes and malodor counteractants, such as cyclodextrin, are known in the art. In the processing of some chemical formulation, such as freshening compositions, silicone anti-foaming agents are used to control and limit the level of foam generated in the making process. Silicone anti-foaming agents may also be used in other fabric and home care compositions, such as laundry and/or automatic dishwashing compositions, to control the amount of foam produced during the washing cycle. Thus, even if not added intentionally, it is possible that silicone anti-foaming agents from a previous production process can be formulated into chemical compositions. However, silicone anti-foaming agents, the silicone anti-foaming agents may interact with other constituents of a freshening compositions, such as cyclodextrin. Thus, it would be beneficial to provide a freshening composition that is able to limit the interaction between silicone anti-foaming agents and constituents such as cyclodextrin.

### SUMMARY

“Combinations:”

A. A freshening composition comprising:

a liquid carrier;

cyclodextrin;

a silicone anti-foam agent, wherein the weight ratio of silicone anti-foam agent to cyclodextrin is less than 0.00375:1.

B. The freshening composition of Paragraph A, wherein the weight ratio of silicone anti-foam agent to cyclodextrin is less than 0.0025:1.

C. The freshening composition of Paragraph A or Paragraph B further comprising a perfume mixture.

D. The freshening composition of Paragraph C, wherein a weight ratio of perfume mixture to cyclodextrin is 0.001:1 to 30:1, more preferably 0.01:1 to 15:1, and more preferably 0.05:1 to 5:1.

E. The freshening composition of any of Paragraphs A through D, wherein the cyclodextrin is present at a level of 0.01 wt. % to 3 wt. %, based on the total weight of the freshening composition.

F. The freshening composition of any of Paragraphs A through E, wherein the liquid carrier is water, and wherein the water is present at a level of 85 wt. % to 99.5 wt. %, based on the total weight of the freshening composition.

G. The freshening composition of any of Paragraphs A through F, wherein the silicone anti-foam agent comprises an organopolysiloxane or organomodified silicone polymer.

H. A consumer product comprising:

a spray dispenser

a freshening composition comprising:
a liquid carrier;
cyclodextrin;
a silicone anti-foam agent, wherein the weight ratio of silicone anti-foam agent to cyclodextrin is less than 0.00375:1.

I. The consumer product of Paragraph H further comprising a propellant.

J. The consumer product of Paragraph H or Paragraph I, wherein the spray dispenser is configured to dispense spray droplets of the freshening composition having a Sauter Mean Diameter of 10 μm to 100 μm.

K. The consumer product of any of Paragraphs H through J, wherein the weight ratio of silicone anti-foam agent to cyclodextrin is less than 0.0025:1.

L. The consumer product of any of Paragraphs H through K further comprising a perfume mixture, and wherein a weight ratio of perfume mixture to cyclodextrin is 0.001:1 to 30:1, more preferably 0.01:1 to 15:1, and more preferably 0.05:1 to 5:1.

M. The consumer product of any of Paragraphs H through L, wherein the cyclodextrin is present at a level of 0.01 wt. % to 3 wt. %, based on the total weight of the freshening composition.

N. The consumer product of any of Paragraphs H through M, wherein the liquid carrier is water, and wherein the water is present at a level of 85 wt. % to 99.5 wt. %, based on the total weight of the freshening composition.

O. The consumer product of any of Paragraphs H through N, wherein the silicone anti-foam agent comprises an organopolysiloxane or organomodified silicone polymer.

### BRIEF DESCRIPTION OF THE DRAWINGS

FIG. 1 is a plot of the % nicotine headspace reduction of various compositions as a function of the presence of cyclodextrin and/or a silicone anti-foam agent.

### DETAILED DESCRIPTION

Freshening compositions of the present invention comprise cyclodextrin, a low level of a silicone anti-foam agent, and a carrier. The freshening compositions may comprise perfume.

Cyclodextrin

The freshening composition of the present invention include cyclodextrin. As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in donut-shaped rings. The specific coupling and conformation of the glucose units give the cyclodextrins a rigid, conical molecular structure with hollow interiors of specific volumes. The “lining” of each internal cavity is formed by hydrogen atoms and glycosidic bridging oxygen atoms; therefore, this surface is more hydrophobic. The unique shape and physical-chemical properties of the cavity enable the cyclodextrin molecules to absorb (form inclusion complexes with) organic molecules or parts of organic molecules which can fit into the cavity. Many odorous molecules can fit into the cavity including many malodorous molecules and perfume molecules. Therefore, cyclodextrins, and especially mixtures of cyclodextrins with different size cavities, can be used to control odors caused by a broad spectrum of organic odoriferous materials, which may, or may not, contain reactive functional groups. The complexation between cyclodextrin and odorous molecules occurs rapidly in the presence of water. However, the extent of the complex formation also depends on the polarity of the absorbed molecules. In an aqueous solution, strongly hydrophilic molecules (those which are highly water-soluble) are only partially absorbed, if at all. Therefore, cyclodextrin does not complex effectively with some very low molecular weight organic amines and acids when they are present at low levels on wet fabrics. As the water is being removed however, e.g., the fabric is being dried off, some low molecular weight organic amines and acids have more affinity and will complex with the cyclodextrins more readily.

The cavities within the cyclodextrin in the solution of the present invention should remain essentially unfilled (the cyclodextrin remains uncomplexed) while in solution, in order to allow the cyclodextrin to absorb various odor molecules when the solution is applied to a surface. Non-derivatised (normal) beta-cyclodextrin can be present at a level up to its solubility limit of about 1.85% (about 1.85 g in 100 grams of water) at room temperature. Beta-cyclodextrin is not preferred in compositions which call for a level of cyclodextrin higher than its water solubility limit. Non-derivatised beta-cyclodextrin is generally not preferred when the composition contains surfactant since it affects the surface activity of most of the preferred surfactants that are compatible with the derivatized cyclodextrins.

The freshening composition of the present invention may be clear. The term “clear” as defined herein means transparent or translucent, preferably transparent, as in “water clear,” when observed through a layer having a thickness of less than about 10 cm.

The cyclodextrins used in the present invention may be highly water-soluble such as, alpha-cyclodextrin and/or derivatives thereof, gamma-cyclodextrin and/or derivatives thereof, derivatised beta-cyclodextrins, and/or mixtures thereof. The derivatives of cyclodextrin consist mainly of molecules wherein some of the OH groups are converted to OR groups. Cyclodextrin derivatives include, e.g., those with short chain alkyl groups such as methylated cyclodextrins, and ethylated cyclodextrins, wherein R is a methyl or an ethyl group; those with hydroxyalkyl substituted groups, such as hydroxypropyl cyclodextrins and/or hydroxyethyl cyclodextrins, wherein R is a —CH₂ —CH(OH)—CH₃, —CH(CH₃)—CH₂—OH, or a —CH₂ CH₂ —OH group; branched cyclodextrins such as maltose-bonded cyclodextrins; cationic cyclodextrins such as those containing 2-hydroxy-3-(dimethylamino)propyl ether, wherein R is CH2 —CH(OH)—CH2 —N(CH3)2 which is cationic at low pH; quaternary ammonium, e.g., 2-hydroxy-3-(trimethylammonio)propyl ether chloride groups, wherein R is CH2 —CH(OH)—CH2 —N+(CH3)3 Cl—; anionic cyclodextrins such as carboxymethyl cyclodextrins, cyclodextrin sulfates, and cyclodextrin succinylates; amphoteric cyclodextrins such as carboxymethyl/quaternary ammonium cyclodextrins; cyclodextrins wherein at least one glucopyranose unit has a 3-6-anhydro-cyclomalto structure, e.g., the mono-3-6-anhydrocyclodextrins, as disclosed in “Optimal Performances with Minimal Chemical Modification of Cyclodextrins”, F. Diedaini-Pilard and B. Perly, The 7th International Cyclodextrin Symposium Abstracts, April 1994, p. 49, said references being incorporated herein by reference; and mixtures thereof.

Highly water-soluble cyclodextrins are those having water solubility of at least about 10 g in 100 ml of water at 25° C., preferably at least about 20 g in 100 ml of water, more preferably at least about 25 g in 100 ml of water at room temperature. The availability of solubilized, uncomplexed cyclodextrins is essential for effective and efficient odor control performance. Solubilized, water-soluble cyclodextrin can exhibit more efficient odor control performance than non-water-soluble cyclodextrin when deposited onto surfaces, especially fabric.

Examples of preferred water-soluble cyclodextrin derivatives suitable for use herein are hydroxypropyl alpha-cyclodextrin, methylated alpha-cyclodextrin, methylated beta-cyclodextrin, hydroxyethyl beta-cyclodextrin, and hydroxypropyl beta-cyclodextrin. Hydroxyalkyl cyclodextrin derivatives preferably have a degree of substitution of from about 1 to about 14, more preferably from about 1.5 to about 7, wherein the total number of OR groups per cyclodextrin is defined as the degree of substitution. Methylated cyclodextrin derivatives typically have a degree of substitution of from about 1 to about 18, preferably from about 3 to about 16. A known methylated beta-cyclodextrin is heptakis-2,6-di-O-methyl-β-cyclodextrin, commonly known as DIMEB, in which each glucose unit has about 2 methyl groups with a degree of substitution of about 14. A preferred, more commercially available, methylated beta-cyclodextrin is a randomly methylated beta-cyclodextrin, commonly known as RAMEB, having different degrees of substitution, normally of about 12.6. RAMEB is more preferred than DIMEB, since DIMEB affects the surface activity of the preferred surfactants more than RAMEB. The preferred cyclodextrins are available, e.g., from Cerestar USA, Inc. and Wacker Chemicals (USA), Inc.

It may be preferable to use a mixture of cyclodextrins. The mixture may include alkoxylated and/or non-alkoxylated cyclodextrins. Such mixtures absorb odors more broadly by complexing with a wider range of odoriferous molecules having a wider range of molecular sizes. Preferably at least a portion of the cyclodextrins is alpha-cyclodextrin and its derivatives thereof, gamma-cyclodextrin and its derivatives thereof, and/or derivatised beta-cyclodextrin, more preferably a mixture of alpha-cyclodextrin, or an alpha-cyclodextrin derivative, and derivatised beta-cyclodextrin, even more preferably a mixture of derivatised alpha-cyclodextrin and derivatised beta-cyclodextrin, most preferably a mixture of hydroxypropyl alpha-cyclodextrin and hydroxypropyl beta-cyclodextrin, and/or a mixture of methylated alpha-cyclodextrin and methylated beta-cyclodextrin.

Suitable levels of cyclodextrin are from about 0.01 wt. % to about 3 wt. %, alternatively from about 0.01 wt. % to about 2 wt. %, alternatively from about 0.05 wt. % to about 1 wt. %, alternatively from about 0.05 wt. % to about 0.5 wt. %, by weight of the composition.

Concentrated compositions can also be used in order to deliver a less expensive product. When a concentrated product is used, i.e., when the level of cyclodextrin used is from about 3% to about 20%, more preferably from about 5% to about 10%, by weight of the concentrated composition, it is preferable to dilute the concentrated composition before treating fabrics in order to avoid staining. Preferably the concentrated cyclodextrin composition is diluted with about 50 wt. % to about 6000 wt. %, more preferably with about 75 wt. % to about 2000 wt. %, most preferably with about 100% to about 1000 wt. % by weight of the concentrated composition of water. The resulting diluted compositions have usage concentrations of cyclodextrin as discussed hereinbefore, e.g., of from about 0.1 wt. % to about 5 wt. %, by weight of the diluted composition.

Silicone Anti-Foam Agent

The freshening compositions of the present invention includes a silicone anti-foam agent. In a freshening compositions comprising cyclodextrin, the weight ratio of silicone anti-foam agent to cyclodextrin may be less than 0.00375:1 or preferably less than 0.0025:1. It has been found that a silicone anti-foam agent present at a weight ratio of silicone anti-foam agent to cyclodextrin of less than 0.00375:1 does not significantly interfere with the ability of cyclodextrin to bind with malodors, while a silicone anti-foam agent present above this ratio interferes with the ability of cyclodextrin to bind with malodors.

The silicone anti-foam agent may be selected from silicone anti-foam compounds; anti-foam compounds of silicone oils and hydrophobic particles; and mixtures thereof. The percentage of silicone anti-foaming agent is expressed as a percentage by active amount not including any carrier.

The anti-foam may be selected from: organomodified silicone polymers or organopolysiloxane material with aryl or alkylaryl substituents combined with silicone resin and modified silica; M/Q resins; and mixtures thereof.

The anti-foam may be selected from organomodified silicone polymers or organopolysiloxane material with aryl or alkylaryl substituents combined with silicone resin and a primary filler.

Particularly preferred are silicone anti-foam compounds consisting of organomodified silicone polymers with aryl or alkyaryl substituents combined with silicone resin and modified silica as described in U.S. Pat. Nos. 6,521,586B1, 6,521,587B1, US Patent Applications 2005/0239908A1, 2007 01673 A1 to Dow Corning Corp. and US Patent Application 2008/0021152 A1 to Wacker Chemie AG.

The organomodified silicone polymer with aryl or alkaryl substituents (in component (i)) is suitably selected from at least one organosilicon compound which has units of the formula Rₐ(R¹O)_{b}R²_{c}SiO_{(4-a-b-c)/2} (I) in which each R can be identical or different and is H or a monovalent, SiC-bonded, optionally substituted, aliphatic hydrocarbon radical and comprises at least one aromatic hydrocarbon radical covalently attached to silicon via aliphatic groups. R1 can be identical or different and is H or a monovalent, optionally substituted hydrocarbon radical which is attached to Si via a carbon ring atom, R2 can be identical or different and is a monovalent, optionally substituted, aromatic hydrocarbon radical which is attached to the silicon atom via a carbon ring atom, a is 0, 1, 2 or 3, b is 0, 1, 2 or 3 and c is 0, 1, 2 or 3, with the proviso that the sum a+b+c is less than or equal to 3, and in 1-100%, preferably in 10-60%, more preferably in 20-40% of all units of the formula (I) per molecule, c is other than 0, and in at least 50% of all of the units of the formula (I) in the organosilicon compound the sum a+b+c is 2.

The silicone resin (component (ii)) is suitably an organopolysiloxane resin made up of units of the formula R³_{d}(R⁴O)_{c}SiO_{(4-d-e)/2} (II) in which R3 can be identical or different and is H or a monovalent, optionally substituted, SiC-bonded hydrocarbon radical. R4 can be identical or different and is H or a monovalent, optionally substituted hydrocarbon radical, d is 0, 1, 2 or 3 and e is 0, 1, 2 or 3, with the proviso that the sum d+e≤3 and in less than 50% of all of the units of the formula (II) in the organopolysiloxane resin the sum d+e is 2,

The anti-foam may further optionally comprise an organosilicon compound which has units of the formula R⁵_{g}(R⁶O)ₕSiO_{(4-g-h)/2} (III) in which R5 can be identical or different and has a meaning given for R, R6 can be identical or different and has a meaning given for R1, g is 0, 1, 2 or 3 and h is 0, 1, 2 or 3, with the proviso that the sum g+h≤3 and in at least 50% of all of the units of the formula (III) in the organosilicon compound the sum g+h is 2.

In one embodiment, the organomodified silicone polymers having aryl or alkaryl substituents component comprises aromatic radicals attached directly to the silicon atom. In such polymers, there is a covalent bond between a silicon atom in the unit of the formula (I) and a carbon atom belonging to the aromatic ring.

The organosilicon compounds containing units of the formula (I) that are used as component (i) are preferably branched or linear organopolysiloxanes which more preferably are composed of units of the formula (I).

In the context of the present invention the term “organopolysiloxanes” is intended to embrace polymeric, oligomeric and dimeric siloxanes.

Primary fillers employed in accordance with the invention may comprise exclusively pulverulent fillers, more preferably pulverulent hydrophobic fillers.

Examples of primary fillers are silicon dioxide (silicas), titanium dioxide, aluminum oxide, metal soaps, quartz flour, PTFE powders, fatty acid amides, ethylenebisstearamide for example, and finely divided hydrophobic polyurethanes.

As primary filler it is possible to use both pretreated silicas, i.e., commercially customary hydrophobic silicas, and hydrophilic silicas.

Organopolysiloxane+Organosilicon Resin+Hydrophobic Filler

Anti-foams useful herein include those silicone anti-foams described in U.S. Pat. Nos. 6,251,586 and 6,251,587, both to Dow Corning. Such anti-foams comprise (A) an organopolysiloxane material having at least one silicon-bonded substituent of the formula X-Ph, wherein X denotes a divalent aliphatic organic group bonded to silicon through a carbon atom and Ph denotes an aromatic group, (B) an organosilicon resin and (C) a hydrophobic filler. The aromatic group can be unsubstituted or substituted.

The organopolysiloxane material (A) is preferably a fluid and is preferably a polydiorganosiloxane. The polydiorganosiloxane (A) preferably comprises diorganosiloxane units of the formula:

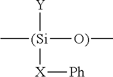

where Y is an alkyl group having 1 to 4 carbon atoms, preferably methyl. These diorganosiloxane units containing a —X-Ph group may comprise substantially all or a majority of the diorganosiloxane units in organopolysiloxane (A), but preferably comprise up to 50 or 60%, most preferably 5 to 40%, of the diorganosiloxane units in (A). The group X is preferably a divalent alkylene group having from 2 to 10 carbon atoms, most preferably 2 to 4 carbon atoms, but can alternatively contain an ether linkage between two alkylene groups or between an alkylene group and -Ph, or can contain an ester linkage. Ph is preferably a moiety containing at least one aromatic ring —C6 R5, wherein each R independently denotes hydrogen, halogen, hydroxyl, an alkoxy group having 1 to 6 carbon atoms or a monovalent hydrocarbon group having 1 to 12 carbon atoms, or wherein two or more R groups together represent a divalent hydrocarbon group. Ph is most preferably a phenyl group, but may be substituted for example by one or more methyl, methoxy, hydroxyl or chloro group, or two substituents R may together form a divalent alkylene group, or may together form an aromatic ring, resulting in conjunction with the Ph group in e.g. a naphthalene group. A particularly preferred X-Ph group is 2-phenylpropyl —CH2-CH(CH3)-C6 H5. Alternatively, Ph can be a heterocyclic group of aromatic character such as thiophene, pyridine or quinoxaline.

The organopolysiloxane material (A) may be a substantially linear polydiorganosiloxane or may have some branching. The branching may be in the siloxane chain, brought about e.g. by the presence of some tri-functional siloxane units of the formula ZSiO3/2, where Z denotes a hydrocarbon, hydroxyl or hydrocarbonoxy group. Alternatively branching may be caused by a multivalent, e.g. divalent or trivalent, organic or silicon-organic moiety linking siloxane polymer chains. The organic moiety can be a divalent linking group of the formula —X′—, and the silicon-organic moiety can be a divalent linking group of the formula X′—Sₓ—X′, where X′ denotes a divalent organic group bonded to silicon through a carbon atom and Sₓ is an organosiloxane group. Examples of organic linking (branching) units are C₂₋₆ alkylene groups, e.g. dimethylene or hexylene, or aralkylene groups of the formula —X′—Ar—X′—, where Ar denotes phenylene. Hexylene units can be introduced by reaction of 1,5-hexadiene with Si—H groups and —X′—Ar—X′— units by reaction of divinylbenzene or diisopropylbenzene. Examples of silicon-organic linking units are those of the formula —(CH₂)_{d}—(Si(CH₃)₂—O)_{c}—Si(CH₃)₂—(CH₂)_{d}— wherein d has a value of from 2 to 6 and e has a value of from 1 to 10; for example linking units of the latter formula with d=2 and e=1 can be introduced by reaction of divinyltetramethyldisiloxane with Si—H groups.

The organosilicon resin (B) is generally a non-linear siloxane resin and preferably consists of siloxane units of the formula R′ₐ SiO₄₋ₐ/2 wherein R′ denotes a hydroxyl, hydrocarbon or hydrocarbonoxy group and wherein a has an average value of from 0.5 to 2.4. The resin preferably consists of monovalent trihydrocarbonsiloxy (M) groups of the formula R″3 SiO₁/2 and tetrafunctional (O) groups SiO₄/2 wherein R″ denotes a monovalent hydrocarbon group. The number ratio of M groups to Q groups is preferably in the range 0.4:1 to 2.5:1 (equivalent to a value of a in the formula R′ₐ SiO₄₋ₐ/2 of 0.86 to 2.15), and is more preferably 0.4:1 to 1.1:1 and most preferably 0.5:1 to 0.8:1 (equivalent to a=1.0-1.33) for use in freshening applications. The organosilicon resin (B) is preferably a solid at room temperature, but MQ resins having a M/Q ratio of higher than 1.2, which are generally liquid, can be used successfully. Although it is most preferred that the resin (B) consists only of M and Q groups as defined above, a resin comprising M groups, trivalent R″SiO₃/2 (T) groups and Q groups can alternatively be used. The organosilicon resin (B) can also contain divalent units R″2 SiO₂/2, preferably at no more than 20% of all siloxane units present. The group R″ is preferably an alkyl group having from 1 to 6 carbon atoms, most preferably methyl or ethyl, or phenyl. It is particularly preferred that at least 80%, and most preferably substantially all of the R″ groups present are methyl groups. Other hydrocarbon groups may also be present, e.g. alkenyl groups present for example as dimethylvinylsilyl units, preferably in small amounts, most preferably not exceeding 5% of all R″ groups. Silicon bonded hydroxyl groups and/or alkoxy, e.g. methoxy, groups may also be present.

The organosilicon resin (B) is preferably present in the anti-foam at 1-50% by weight based on organopolysiloxane (A), particularly 2-30% and most preferably 4-15%.

The organosilicon resin (B) may be soluble or insoluble (not wholly dissolved) in the organopolysiloxane (A) when present in the above amounts. Solubility can be measured by observing a mixture of (A) and (B) in an optical microscope. The compositions may contain dissolved organosilicon resin (B) and by compositions containing dispersed particles of organosilicon resin (B). The factors affecting solubility of (B) in (A) include the proportion of X-Ph groups in (A) (more X-Ph groups increase solubility), the degree of branching in (A), the nature of the groups Y and Y′ in (A) (long chain alkyl groups decrease solubility), the ratio of M to Q units in MQ resin (B) (higher ratio of M groups to Q groups increases solubility) and the molecular weight of (B). The solubility of (B) in (A) at ambient temperature can thus be from 0.01% by weight or less up to 15% or more. It may be advantageous to use a mixture of a soluble resin (B) and an insoluble resin (B), for example a mixture of MQ resins having different M/Q ratios. If the organosilicon resin (B) is insoluble in organopolysiloxane (A), the average particle size of resin (B), as measured when dispersed in liquid (A), may for example be from 0.5 to 400 μm, preferably 2 to 50 μm. For industrial foam control applications such as defoaming of black liquor in the paper and pulp industry, resins which are soluble in the siloxane copolymer, such as MQ resins having a high M/Q ratio, are usually preferred.

The silicone anti-foam agent may include a hydrophobic filler (C). Hydrophobic fillers for anti-foams are well known and may be such materials as silica, preferably with a surface area as measured by BET measurement of at least 50 m2/g, titania, ground quartz, alumina, aluminosilicates, organic waxes e.g. polyethylene waxes and microcrystalline waxes, zinc oxide, magnesium oxide, salts of aliphatic carboxylic acids, reaction products of isocyanates with certain materials, e.g. cyclohexylamine, or alkyl amides, e.g. ethylenebisstearamide or methylenebisstearamide. Mixtures of one or more of these are also acceptable.

Exemplary silicone anti-foam agents include XIAMETER(R) AFE-0020 ANTIFOAM EMULSION, available from The Dow Chemical Company, XIAMETER(R) AFE-1520 ANTIFOAM EMULSION, available from The Dow Chemical Company, and AF-8017 Antifoam, available from The Dow Chemical Company.

Perfume Mixture

The freshening composition also includes a perfume mixture comprising at least one perfume raw materials (PRMs). Various PRMs may be used. The perfume mixture may comprise one or more of the following perfume raw materials: fragrant essential oils; natural and synthetic aroma compounds; pro-perfumes; materials supplied with the fragrant essential oils, aroma compounds, and/or pro-perfumes, including stabilizers, diluents, processing agents, and contaminants; and any material that commonly accompanies fragrant essential oils, aroma compounds, and/or pro-perfumes.

The perfume mixture may comprise about 0.01 wt. % to about 10 wt. %, alternatively about 0.01 wt. % to about 5 wt. %, alternatively about 0.01 wt. % to about 3 wt. %. Concentrated compositions may include higher concentrations of the perfume mixture.

If a perfume mixture is present, the perfume mixture to cyclodextrin weight ratio may be from about 0.001:1 to about 30:1, preferably from about 0.01:1 to about 15:1, more preferably from about 0.05:1 to about 5:1, even more preferably from about 0.05:1 to about 1:1, most preferably from about 0.05:1 to about 0.5:1.

Buffer System

The freshening composition may include a buffering agent. The buffering agent may be an acidic buffering agent. The buffering agent may be a dibasic acid, carboxylic acid, dicarboxylic acid such as maleic acid, tricarboxylic acid such as citric acid, or a polycarboxylic acid such as polyacrylic acid. The carboxylic acid may be, for example, citric acid, polyacrylic acid, or maleic acid. The acid may be sterically stable. The acid may be used in the composition for maintaining the desired pH. The freshening composition may have a pH from about 4 to about 9, alternatively from about 4 to about 8.5, alternatively from about 4 to about 6.9, alternatively about 4 to about 6.7.

Preferably, the buffer system comprises one or more buffering agents selected from the group consisting of: citric acid, maleic acid, polyacrylic acid, and combinations thereof. It has been found that buffer systems that include a buffering agent selected from the group consisting of: citric acid, maleic acid, polyacrylic acid, and combinations thereof provide stable freshening compositions with prolonged shelf life.

Preferably, the buffer system comprises citric acid and sodium citrate. It has been found that buffer systems comprising citric acid and sodium citrate provide stable freshening compositions with a prolonged shelf life.

Other suitable buffering agents for the freshening compositions include biological buffering agents. Some examples are nitrogen-containing materials, sulfonic acid buffers like 3-(N-morpholino)propanesulfonic acid (MOPS) or N-(2-Acetamido)-2-aminoethanesulfonic acid (ACES), which have a near neutral 6.2 to 7.5 pKa and provide adequate buffering capacity at a neutral pH. Other examples are amino acids such as lysine or lower alcohol amines like mono-, di-, and tri-ethanolamine or methyldiethanolamine or derivatives thereof. Other nitrogen-containing buffering agents are tri(hydroxymethyl)amino methane (HOCH2)3CNH3 (TRIS), 2-amino-2-ethyl-1,3-propanediol, 2-amino-2-methyl-propanol, 2-amino-2-methyl-1,3-propanol, disodium glutamate, N-methyl diethanolamide, 2-dimethylamino-2-methylpropanol (DMAMP), 1,3-bis(methylamine)-cyclohexane, 1,3-diamino-propanol N,N′-tetra-methyl-1,3-diamino-2-propanol, N,N-bis(2-hydroxyethyl)glycine (bicine) and N-tris (hydroxymethyl)methyl glycine (tricine). Mixtures of any of the above are also acceptable.

The freshening compositions may include a secondary or tertiary amine

The freshening compositions may contain at least about 0%, alternatively at least about 0.001%, alternatively at least about 0.01%, by weight of the composition, of a buffering agent. The composition may also contain no more than about 2%, alternatively no more than about 0.75%, alternatively no more than about 0.5%, by weight of the composition, of a buffering agent.

Solubilizer

The freshening composition may contain a solubilizing aid to solubilize any excess hydrophobic organic materials, particularly any PRMs, and also optional ingredients (e.g., insect repelling agent, antioxidant, etc.) which can be added to the composition, that are not readily soluble in the composition, to form a clear solution. A suitable solubilizing aid is a surfactant, such as a no-foaming or low-foaming surfactant. Suitable surfactants are anionic surfactants, nonionic surfactants, cationic surfactants, amphoteric surfactants, zwitterionic surfactants, and mixtures thereof.

The freshening composition may contain nonionic surfactants, cationic surfactants, and mixtures thereof. The freshening composition may contain surfactant derivatives of hydrogenated castor oil. Suitable ethoxylated hydrogenated castor oils that may be used in the present composition include BASOPHOR™, available from BASF, and CREMOPHOR™, available from Sigma Aldrich.

When the solubilizing agent is present, it is typically present at a level of from about 0.01% to about 3%, alternatively from about 0.05% to about 1%, alternatively from about 0.01% to about 0.05%, by weight of the freshening composition.

Wetting Agent

The freshening composition may, optionally, include a wetting agent that provides a low surface tension that permits the composition to spread readily and more uniformly on hydrophobic surfaces like polyester and nylon. It has been found that the freshening composition, without such a wetting agent will not spread satisfactorily. The spreading of the composition also allows it to dry faster, so that the treated material is ready to use sooner. Furthermore, a composition containing a wetting agent may penetrate hydrophobic, oily soil better for improved malodor neutralization. A composition containing a wetting agent may also provide improved “in-wear” electrostatic control. For concentrated compositions, the wetting agent facilitates the dispersion of many actives such as antimicrobial actives and perfumes in the concentrated freshening compositions.

Non-limiting examples of wetting agents include block copolymers of ethylene oxide and propylene oxide. Suitable block polyoxyethylene-polyoxypropylene polymeric surfactants include those based on ethylene glycol, propylene glycol, glycerol, trimethylolpropane and ethylenediamine as the initial reactive hydrogen compound. Polymeric compounds made from a sequential ethoxylation and propoxylation of initial compounds with a single reactive hydrogen atom, such as C₁₂₋₁₈ aliphatic alcohols, are not generally compatible with the cyclodextrin.

Certain of the block polymer surfactant compounds designated Pluronic® and Tetronic® by the BASF-Wyandotte Corp., Wyandotte, Mich., are readily available.

Non-limiting examples of cyclodextrin-compatible wetting agents of this type are described in U.S. Pat. No. 5,714,137 and include the SILWET® surfactants available from Momentive Performance Chemical, Albany, N.Y. Exemplary SILWET surfactants are as follows:

| Name | Average MW |
|---|---|
| L-7608 | 600 |
| L-7607 | 1,000 |
| L-77 | 600 |
| L-7605 | 6,000 |
| L-7604 | 4,000 |
| L-7600 | 4,000 |
| L-7657 | 5,000; |

and mixtures thereof.

The total amount of surfactants (e.g. solubilizer, wetting agent) in the freshening composition is from 0 wt. % to about 3 wt. % or no more than 3 wt. %, alternatively from 0 wt. % to about 1 wt. % or no more than 1 wt. %, alternatively from 0 wt. % to about 0.9 wt. % or no more than 0.9 wt. %, alternatively from 0 wt. % to about 0.7 wt. % or no more than 0.7 wt. %, alternatively from 0 wt. % to about 0.5 wt. % or no more than 0.5 wt. %, alternatively from 0 wt. % to 0.3 wt. % or no more than about 0.3 wt. %, by weight of the composition. Compositions with higher concentrations can make fabrics susceptible to soiling and/or leave unacceptable visible stains on fabrics as the solution evaporates.

The weight ratio of sulfur-containing pro-perfume to total surfactant may be from about 1:1 to about 1:60, or from about 1:1 to about 1:30.

Carrier

The liquid composition includes a carrier. The carrier which is used may be water. The water may be distilled, deionized, tap, or further purified forms of water. Water may be present in any amount for the composition to be an aqueous solution. Water may be present in an amount from about 80 wt. % to 99.5 wt. %, alternatively from about 85 wt. % to about 99.5 wt. %, alternatively from about 92 wt. % to about 99.5 wt. %, alternatively from about 95 wt. %, by weight of said freshening composition.

Low molecular weight monohydric alcohols (e.g., ethanol, methanol, and isopropanol, or polyols, such as ethylene glycol and propylene glycol) can also be useful either alone or in combination with water.

In some instances, the level of monohydric alcohol may be less than about 20 wt. %, or less than about 15 wt. %, or less than about 10 wt. %, or less than about 6 wt. %, alternatively less than about 3 wt. %, alternatively less than about 1 wt. %, by weight of the freshening composition.

Adjuvants can be optionally added to the freshening composition herein for their known purposes. Such adjuvants include, but are not limited to, water soluble metallic salts, antistatic agents, insect and moth repelling agents, colorants, antioxidants, and mixtures thereof.

Other Ingredients

In addition to, or in place of, perfume materials, the freshening composition may include preservatives; antimicrobial compounds; materials that acts to condition, modify, or otherwise modify the environment (e.g. to assist with sleep, wake, respiratory health, and like conditions); deodorants or malodor control compositions (e.g. odor neutralizing materials such as reactive aldehydes (as disclosed in U.S. 2005/0124512), odor blocking materials, odor masking materials, or sensory modifying materials such as ionones (also disclosed in U.S. 2005/0124512)), and other malodor removal compounds such as polyamines, including polyethylene imines Such adjuvants include, but are not limited to, water soluble metallic salts, antistatic agents, insect and moth repelling agents, colorants, antioxidants, and mixtures thereof.

Freshening Product

The freshening composition can be packaged in any suitable package to form a freshening product. The package may be in the form of a spray dispenser.

The spray dispenser may be transparent or translucent such that the freshening composition is visible or at least partially visible from outside of the freshening product.

The spray dispenser may be comprised of various materials, including plastic, metal, glass, or combinations thereof. The spray dispenser may be pressurized or unpressurized.

One suitable spray dispenser is a plastic aerosol dispenser. The dispenser may be constructed of polyethylene such as a high-density polyethylene; polypropylene; polyethyleneterephthalate (“PET”); vinyl acetate, rubber elastomer, and combinations thereof. The spray dispenser may be made of clear PET.

Another suitable spray dispenser includes a continuous action sprayer, such as FLAIROSOL™ dispenser from Afa Dispensing Group. The FLAIROSOL™ dispenser includes a bag-in-bag or bag-in-can container with a pre-compression spray engine, and aerosol-like pressurization of the freshening composition.

The spray dispenser may hold various amounts of freshening composition.

The spray dispenser may be capable of withstanding internal pressure in the range of about 20 p.s.i.g. to about 140 psig, alternatively about 80 to about 130 p.s.i.g.

The total composition output and the spray droplet/particle size distribution may be selected to support the particulate removal efficacy but avoid a surface wetness problem. Total output is determined by the flow rate of the composition as it is released from the spray dispenser. To achieve a spray profile that produces minimal surface wetness, it is desirable to have a low flow rate and small spray droplets.

The flow rate of the composition being released from the spray dispenser may be from about 0.0001 grams/second (g/s) to about 2.5 grams/second. Alternatively, the flow rate may be from about 0.001 grams/second to about 2.5 grams/second, or about 0.01 grams/second to about 2.0 grams/second. For an aerosol sprayer, the flow rate is determined by measuring the rate of composition expelled by a spray dispenser for any 60 second period of use.

The Sauter Mean Diameter of the spray droplets may be in the range of from about 10 μm to about 100 μm, alternatively from about 20 μm to about 60 μm. At least some of the spray droplets are sufficiently small in size to be suspended in the air for at least about 10 minutes, and in some cases, for at least about 15 minutes, or at least about 30 minutes.

Small particles can be efficiently created when the spray is dispensed in a wide cone angle. For a given nozzle component and delivery tube, cone angles can be modified by varying the insertion depth of the nozzle in the delivery tube. The cone angle may be greater than about 20 degrees, or greater than about 30 degrees, or greater than about 35 degrees, or greater than about 40 degrees, or greater than about 50 degrees.

The spray dispenser may be configured to spray the composition at an angle that is between an angle that is parallel to the base of the container and an angle that is perpendicular thereto. The desired size of spray droplets can be delivered by other types of spray dispensers that are capable of being set to provide a narrow range of droplet size. Such other spray dispensers include, but are not limited to: foggers, ultrasonic nebulizers, electrostatic sprayers, and spinning disk sprayers.

A pressurized spray dispenser may include a propellant. Various propellants may be used. The propellant may comprise hydrocarbon(s); compressed gas(es), such as nitrogen, carbon dioxide, air; liquefied gas(es) or hydrofluoro olefin (“HFO”); and mixtures thereof. Preferably, the product comprises a propellant selected from the group consisting of compressed gas such as compressed air, compressed nitrogen, and combinations thereof. Propellants listed in the U.S. Federal Register 49 C.F.R. § 1.73.115, Class 2, Division 2.2 are considered acceptable. The propellant may particularly comprise a trans-1,3,3,3-tetrafluoroprop-1-ene, and optionally a CAS number 1645-83-6 gas. Such propellants provide the benefit that they are not flammable, although the freshening compositions are not limited to inflammable propellants. One such propellant is commercially available from Honeywell International of Morristown, N.J. under the trade name HFO-1234ze or GWP-6.

If desired, the propellant may be condensable. By “condensable”, it is meant that the propellant transforms from a gaseous state of matter to a liquid state of matter in the spray dispenser and under the pressures encountered in use. Generally, the highest pressure occurs after the spray dispenser is charged with a freshening composition but before that first dispensing of that freshening composition by the user. A condensable propellant provides the benefit of a flatter depressurization curve as the freshening composition is depleted during usage.

The pressurized spray dispenser may be free of a hydrocarbon propellant.

The freshening composition may be delivered from the spray dispenser which includes delivery components including but not limited to a valve to control flow and to seal the freshening composition within the spray dispenser, a button actuator and a nozzle for dispensing the freshening composition to the environment.

The liquid composition may be contained in a bag-in-can plastic spray dispenser.

Preferably the freshening composition has b* value of less 40, more preferably less than 20, more preferably less than 14, more preferably less than 20, more preferably less than 7, more preferably less than 5, and most preferably less than 1, according to the methodology in the Example section below. Preferably the freshening composition has a change in b* value, which comparing aged versus initial b* values, of less than 20, preferably less than 14, preferably less than 5.0, more preferably a change of less than 1, and most preferably a change of less than 0.5.

Methods of Use

The freshening composition can be used by dispersing, e.g., by placing the freshening composition into a dispenser, such as a spray dispenser and spraying an effective amount into the air or onto the desired inanimate surface or article. “Effective amount”, when used in connection with the amount of the freshening composition, means an amount sufficient to provide at least about 4 hours, or at least about 6 hours, or at least about 8 hours, or at least about 24 hours of freshness or scent to the treated air, surface, or article, yet not so much as to saturate or create a pool of liquid on an article or surface and so that, when dry, there is no visual deposit readily discernible. Where malodor reducing ingredients are included, “effective amount”, when used in connection with the amount of the freshening composition, means an amount that provides the foregoing and also provides neutralization of a malodor to the point that it is not discernible by the human sense of smell, yet not so much as to saturate or create a pool of liquid on an article or surface and so that, when dry, there is no visual deposit readily discernible. Dispersing can be achieved by using a spray device, a roller, a pad, etc.

### Examples

Method for Measuring Smoke Malodor Reduction Efficacy in Fabric Headspace

1. Circular fabric swatches with a 2 cm diameter were cut from a 50/50 polycotton knit fabric swatch.

2. Fabrics were inoculated with smoke malodor (“Nicotine”) via spiking using an Eppendorf pipette and allowed to dry for 10 minutes. Repetitions of three fabrics per leg were prepared.

3. Fabrics were treated with a product formulation of Table 1 and allowed to dry for 2 hours.

4. Fabric samples were placed into 20 mL headspace vials, capped, and equilibrated at room temperature for at least 2 hours prior to analysis.

5. Headspace samples were analyzed via 6890/5973 GC/MS using MPS-2 autosampler SPME with a gray DVB/CAR/PDMS fiber.

6. Method included a 10-minute extraction at 40° C., followed by sample injection into the GC/MS inlet.

7. The GC/MS method included the following parameters:

a. Oven temperature program: 50° C. with no hold, temperature ramp of 16° C./minute to 280° C. for 3-minute hold, total run time of 17.38 minutes;

b. Split injection at a split ratio of 30:1, inlet at 250° C., and pressure of 65.59 psi;

c. Constant column flow, 0.5 mL/min;

d. Mass spectral data was collected with a scan mass range from 35 to 350.

8. Percent headspace reduction of nicotine was determined by FID for both samples and standards. Commercial or in-house MS library can be used for component identification.

### Example Formulations

| | CD | Example | Example | Example | Comparative | Comparative |
|---|---|---|---|---|---|---|
| | Control | A | B | C | Example D | Example E |
| | | AFE 0020 | AFE 0020 | AFE 0020 | AFE 0020 | AFE 0020 |
| | | Suds | Suds | Suds | Suds | Suds |
| | | Suppressor | Suppressor | Suppressor | Suppressor | Suppressor |
| | | | | | | (premixed |
| | | | | | | with water + |
| | | | | | | 3% ethanol) |
| CD (of the | 3600 | 3600 ppm | 3600 ppm | 3600 ppm | 3600 ppm | 0 |
| total | ppm | | | | | |
| composition) | | | | | | |
| Concentration | 0 | 0.9 ppm | 4.5 ppm | 9.0 ppm | 18 ppm | 18 ppm |
| of silicone | | | | | | |
| anti-foam | | | | | | |
| agent (of the | | | | | | |
| total | | | | | | |
| composition) | | | | | | |
| Weight ratio | 0 | 0.00025:1 | 0.00125:1 | 0.0025:1 | 0.005:1 | 0 |
| of silicone | | | | | | |
| anti-foam | | | | | | |
| agent to | | | | | | |
| cyclodextrin | | | | | | |
| Ethanol | 3.00 | 3.00 wt. % | 3.00 wt. % | 3.00 wt. % | 3.00 wt. % | 3.03 wt. % |
| | wt. % | | | | | |
| Water | balance | balance | balance | balance | balance | balance |

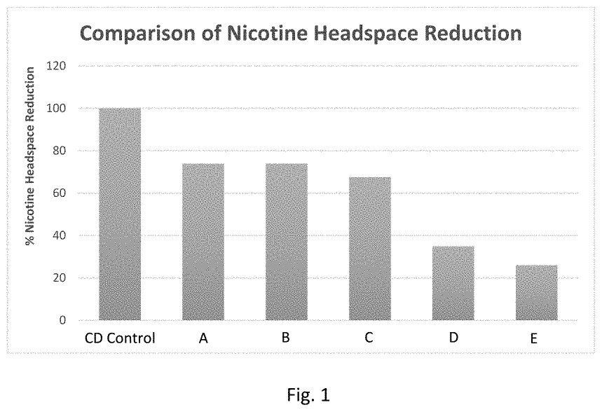

As shown in FIG. 1, the presence of a silicone anti-foam agent in a freshening composition at a weight ratio of silicone anti-foam agent to cyclodextrin below 0.00375:1 impedes but does not affect the majority of the cyclodextrin benefit in reducing a malodor such as a nicotine odor. However, a weight ratio of silicone anti-foam agent to cyclodextrin above 0.00375:1 significantly reduces the malodor benefit that cyclodextrin provides to the composition. In fact, a weight ratio of silicone anti-foam agent to cyclodextrin of 0.005:1 had nearly the same percentage of nicotine odor reduction as a composition having the same concentration of silicone anti-foam agent that did not have cyclodextrin present. Thus, a silicone anti-foam agent, which is used in many chemical manufacturing processes for various reasons, may be present, but needs to be controlled in a freshening composition comprising cyclodextrin at a weight ratio of silicone anti-foam agent to cyclodextrin below 0.00375:1 or more preferably below 0.0025:1.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A freshening composition comprising:
a liquid carrier;
cyclodextrin;
a silicone anti-foam agent, wherein the weight ratio of silicone anti-foam agent to cyclodextrin is less than 0.00375:1.

2. The freshening composition of claim 1, wherein the weight ratio of silicone anti-foam agent to cyclodextrin is less than 0.0025:1.

3. The freshening composition of claim 1 further comprising a perfume mixture.

4. The freshening composition of claim 3, wherein the perfume mixture is present at a level of about 0.01 wt. % to about 10 wt. %, by weight of the total weight of the freshening composition.

5. The freshening composition of claim 3, wherein a weight ratio of perfume mixture to cyclodextrin is about 0.001:1 to about 30:1.

6. The freshening composition of claim 1, wherein the cyclodextrin is present at a level of about 0.01 wt. % to about 3 wt. %, based on the total weight of the freshening composition.

7. The freshening composition of claim 1, wherein the liquid carrier is water, and wherein the water is present at a level of 85 wt. % to 99.5 wt. %, based on the total weight of the freshening composition.

8. The freshening composition of claim 1, wherein the silicone anti-foam agent comprises an organopolysiloxane or organomodified silicone polymer.

9. The freshening composition of claim 1, further comprising a malodor counteractant.

10. A consumer product comprising:
a spray dispenser
a freshening composition comprising:
a liquid carrier;
cyclodextrin;
a silicone anti-foam agent, wherein the weight ratio of silicone anti-foam agent to cyclodextrin is less than 0.00375:1.

11. The consumer product of claim 10 further comprising a propellant.

12. The consumer product of claim 11, wherein the propellant is selected from the group consisting of: compressed gas, hydrocarbon, hydrofluorocarbons, and combinations thereof.

13. The consumer product of claim 10, wherein the spray dispenser is configured to spray droplets of the freshening composition having a Sauter Mean Diameter of about 10 μm to about 100 μm.

14. The consumer product of claim 10, wherein the weight ratio of silicone anti-foam agent to cyclodextrin is less than 0.0025:1.

15. The consumer product of claim 10 further comprising a perfume mixture.

16. The consumer product of claim 15, wherein the perfume mixture is present at a level of about 0.01 wt. % to about 10 wt. %, by weight of the total weight of the freshening composition.

17. The consumer product of claim 10 further comprising a malodor counteractant.

18. The consumer product of claim 10, wherein the cyclodextrin is present at a level of about 0.01 wt. % to about 3 wt. %, based on the total weight of the freshening composition.

19. The consumer product of claim 10, wherein the liquid carrier is water, and wherein the water is present at a level of 85 wt. % to 99.5 wt. %, based on the total weight of the freshening composition.

21. The consumer product of claim 9, wherein the silicone anti-foam agent comprises an organopolysiloxane or organomodified silicone polymer.

20. The consumer product of claim 15, wherein weight ratio of perfume mixture to cyclodextrin is about 0.001:1 to about 30:1.
